SEQUENCE LISTING (8 sequences)

SEQ ID NO: 1 - DNA, 10 bp, Unknown
  Synthesized
tttaaacacc

SEQ ID NO: 2 - DNA, 10 bp, Unknown
  Synthesized
gggaaacacc

SEQ ID NO: 3 - DNA, 24 bp, Unknown
  Synthesized
ctcgtcgctaccgtcgtgacttcg

SEQ ID NO: 4 - DNA, 25 bp, Unknown
  Synthesized
cagatgccggttcaggtactcagtc

SEQ ID NO: 5 - DNA, 16 bp, Unknown
  Synthesized
agagctgaacgggaag

SEQ ID NO: 6 - DNA, 16 bp, Unknown
  Synthesized
gttgaagtcgcaggag

SEQ ID NO: 7 - DNA, 21 bp, Unknown
  Synthesized
ccgagattgagcaataacagg

SEQ ID NO: 8 - DNA, 20 bp, Unknown
  Synthesized
agttcgaccgtcttctcagg

(19) United States
(12) Patent Application Publication
(10) Pub. No.: US 2006/0135612 A1
(43) Pub. Date: Jun. 22, 2006

(54) Method of ameliorating or abrogating the effects of a neurodegenerative disorder, such as amyotrophic lateral sclerosis (ALS), by using a HDAC inhibiting agent

(75) Inventor: Robert J. Ferrante, Canton, MA (US)

Correspondence Address: DINESH AGARWAL, P.C., 5350 SHAWNEE ROAD, SUITE 330, ALEXANDRIA, VA, 22312, US

(73) Assignee: U.S. Department of Veterans Affairs

(21) Appl. No.: 11/304,612

(22) Filed: Dec. 16, 2005

Related U.S. Application Data
(60) Provisional application No. 60/636,489, filed on Dec. 17, 2004.

(51) Int. Cl. A61K 31/19; A61K 31/192

(52) U.S. Cl. 514/557; 514/570

(57) ABSTRACT

A method of ameliorating or abrogating the effects of a neurodegenerative disorder, such as amyotrophic lateral sclerosis (ALS), includes administering a histone deacetylase (HDAC) inhibitor in a subject in need thereof. The HDAC inhibitor includes sodium butyrate or sodium phenylbutyrate.

### CROSS-REFERENCE TO RELATED APPLICATIONS

The present application claims priority on prior U.S. Provisional Application Ser. No. 60/636,489, filed Dec.ember 17, 2004, which is hereby incorporated herein in its entirety by reference.

### STATEMENT REGARDING FEDERALLY SPONSORED RESEARCH OR DEVELOPMENT

The work leading to the present invention was supported by one or more grants from the U.S. Government, including NIH Grant(s): AG 13846, AG 12992, NS 31248, and NS 37912, and the Veterans Administration. The U.S. Government therefore has certain rights in the invention.

### REFERENCE TO SEQUENCE LISTING

The present application incorporates by reference a file named: US 1450-05 Ferrante.ST25, including SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8, provided herewith in a computer readable form—on a diskette, created on Dec. 14, 2005 and containing 2,140 bytes. The sequence listing information recorded on the diskette is identical to the written (on paper) sequence listing provided herein.

### FIELD AND BACKGROUND OF THE INVENTION

Amyotrophic lateral sclerosis (ALS) is a clinically severe and progressively fatal neurodegenerative disorder characterized by a loss of both upper and lower motor neurons, resulting in progressive muscle wasting and subsequent paralysis (Reference 1). The incidence of ALS is approximately 2/100,000/year and may be rising. Death occurs within 2-5 years of diagnosis. Current medical care focuses on symptom management. Supportive care ameliorates symptoms and makes ALS more manageable for patients and their families but does not affect the primary disease process. Riluzole, the only FDA-approved ALS therapy, is associated with a only a 2-3 month prolongation of survival (References 2 and 3).

Missense mutations in the enzyme copper/zinc superoxide dismutase (SOD1) are associated with 15-20% of familial ALS cases (Reference 4). The similarity in the course and pathological features of familial and sporadic ALS has prompted the view that all forms of the disease may be better understood and ultimately trea ted by elucidating disease pathogenesis and developing effective therapeutics using transgenic mouse and rat models of ALS expressing mutant forms of SOD1 (References 5 and 6). These transgenic models develop clinical and pathological features that are strikingly similar to those in the human disease (References 7-9). A small set of beneficial therapeutic trials in transgenic ALS mice have generated trials of potential treatments in humans with both sporadic and familial ALS (References 10-13).

While mechanism(s) precipitating neuronal death in ALS are not fully defined, it is now clear that apoptotic cell death is a common cellular event in both animal models and in patients with ALS (References 14-16). The potential importance of apoptotic death pathways in this disease is suggested by the observations that increased expression of bcl-2, dominant negative inhibition of caspase-1, and administration of tetrapeptide caspase inhibitors significantly delays disease onset and prolongs survival in ALS mice (References 17-19).

Recent evidence suggests that transcriptional dysregulation may play a role in the pathogenesis of ALS (References 20-24). Microarray analysis shows distinct changes in the molecular signature of gene expression in both animal models and patients with ALS (References 20, 21 and 25). Upregulation in TAFII30, one of the TATA-binding protein-associated factors required for transcription of a subset of genes, may alter the activity of cellular transcription, and contribute to neuronal toxicity in ALS (Reference 21).

Transcription is regulated by complex interactions between many proteins, among them transcription factors and histones that ultimately affect the actions of RNA polymerase II on individual genes. Many of these interactions, in turn, are regulated by covalent modifications of, such as acetylation, methylation, and phosphorylation. Recruitment of histone deacetylases to DNA alters nucleosome structure locally and inhibits transcription. Histone deactecylase (HDAC) inhibitors increase acetylation, thereby regulating histones and transcription factors, promoting transcriptional activation. They are selective, however, altering only 2-5% of the genes (Reference 26). Of the five classes of HDAC inhibitors, the butyrates are clinical efficacious and the most widely-studied of the compounds that are able to cross the blood brain barrier (References 27-28).

Neuroprotective therapies that target specific molecular mechanisms have the potential to delay the onset and slow the progression of disease in ALS. Thus, therapies that correct aberrant gene transcription might ameliorate the disease course in ALS. HDAC inhibitors are neuroprotective in Huntington's disease mice and are under investigation in Huntington's disease patients (Reference 29 and U.S. Provisional Patent Appl. No. 60/545,532, filed Feb. 19, 2004).

We, therefore, investigated the effects of sodium butyrate and sodium phenylbutyrate on the clinical and neuropathological characteristics, histone acetylation, programmed cell death, and gene transcription in the G93A transgenic mouse model of ALS.

### BRIEF SUMMARY OF THE INVENTION

Multiple molecular defects trigger cell death in amyotrophic lateral sclerosis (ALS). Among these, altered transcriptional activity may perturb multiple cellular functions, leading to a cascade of secondary pathological effects. We show herein that pharmacological treatment using the histone deacetylase (HDAC) inhibitors sodium butyrate and sodium phenylbutyrate significantly extended survival and improved both the clinical and neuropathological phenotypes in G93A transgenic ALS mice. Butyrate administration ameliorated histone hypoacetylation and induced expression of PIKB and NF-κB p50, and bcl-2, while reducing cytochrome c and caspase expression. Mutational inactivation of κB and curcumin, an NF-κB inhibitor, blocked butyrate-induced bcl-2 promoter activity. We provide evidence that the pharmacological induction of NF-κB-dependent transcription and bcl-2 gene expression can be neuroprotective in ALS by inhibiting programmed cell death.

### BRIEF DESCRIPTION OF THE DRAWINGS

The objects, novel features and advantages of the present invention will become apparent from the following detailed description of the invention, as illustrated in the drawings, in which:

FIG. 1: Improvement of clinical phenotype of the G93A ALS mice using both sodium butyrate (NAB) and phenylbutyrate (PBA). Survival was significantly extended in a dose dependent manner using both sodium butyrate and phenylbutrate (a and b). Both motor performance (c and d), as measured by rotarod, and body weight (e) were significantly improved. In addition, stride length was reduced in the untreated G93A mice, as compared to the PBA-treated mice (f and g). PBA-treated and PBS (untreated) G93A mice at 120 days showed marked hindlimb muscle wasting in the untreated mouse (h).

FIG. 1A: PBA protects neuronal cells from oxidative stress-induced cell death. (a) PBA protects mtSOD1 (G41D) N2a cells as well as WT and WT-SOD1 N2a cells against xanthine/xanthine oxidase (X/XO)-induced cell death. Viability was measured by MTT assay. *, Significantly different from non-treated cells. * P<0.01. (b) N2a cells treated with PBA show increased immunofluorescence staining of cytosolic phosphorylated lκB and translocalization of NF-κB p50 to the nucleus.

FIG. 2: PBA and NAB are neuroprotective in G93A ALS mice: Neuropathological analysis at 120 days of survival showed significant gross atrophy, increased astrogliosis, and neuronal loss in the lumbar spinal cord with significantly reduced neuropathological sequelae in the NAB and PBA treated G93A mice, as compared to wild type (WT) littermate controls. Nissl stained tissue sections from the lumbar spinal cord from WT littermate control (a and e), PBA-treated G93A (b and f), NAB-treated G93A mice (c and g), and untreated G93A mice (d and h) show marked gross atrophy and neuronal loss in the untreated G93A mice, with relative sparing in the butyrate treated G93A mice, as compared to control. There is reduced reactive astrogliosis, GFAP immunostaining, in both the PBA (j) and NAB (k) treated mice, in comparison to untreated mice (l) and in contrast with control (i). Bars in 2d and 2i equal 500 μm and 100 μm, respectively.

FIG. 3: PBA reduces cytochrome c release, caspase-9 and -3 activation and enhances histone acetylation in G93A ALS mice. In comparison to wild type (WT) littermate control mice (a), immunohistochemical evidence shows a dramatic reduction in the release of cytochrome c into the cytosol of neurons in the PBA-treated G93A spinal cord (b), as compared with PBS (vehicle)-treated G93A mice (c). There is reduced expression of activated caspase 9 (d-f) and caspase 3 (g-i) in PBA-treated G93A mice at 120 days of age. Western blot analysis shows that PBA treatment of the G93A mice significantly reduces activated caspase 9 and 3, in comparison to untreated mice (m). Hypoacetylation of histone 4 was present in G93A mice at 120 days of age (l), in comparison to wild type littermate control (j), with increased histone acetylation to near normal levels in PBA-treated G93A mice (k). (n) Western blot of acetylated H3 and H4 confirmed the histopathological findings with markedly increased histone 3 and histone 4 acetylation in PBA-treated WT and G93A mice. Bars in 3a and 3d equal 100 μm.

FIG. 4: PBA induces PlκB and NF-κB p50 expression, and restores bcl-2 levels in G93A ALS mice. Immunohistochemical evidence shows increased phosphorylation of lκB in the ventral horn of PBA-treated G93A mice to normal levels (a-c). There is reduced plκB in vehicle-treated G93A mice, in comparison to WT mice. Marked increased expression of NF-κBp50, especially within the neuronal nuclei, is observed in PBA-treated G93A mice, as compared to WT and vehicle-treated G93A mice (d-f). Bcl-2 expression is restored to WT levels in spinal cord sections (g-i) in PBA-treated G93A mice (h), as compared to vehicle-treated G93A mice (i). (j) Western analysis confirmed the histopathological findings with increased plκB, NF-κB p50 and restored bcl-2 protein levels, as a result of PBA treatment. (k) Bcl-2 mRNA was significantly upregulated in PBA-treated G93A primary tissue culture, as compared to PBS (vehicle). Bar in 4a equals 100 μm.

FIG. 5: PBA increases NF-κBp50 acetylation and bcl-2 mRNA and protein levels in ex-vivo and NF-κB-dependent bcl-2 gene reporter activity in vitro. (a) PBA induced NF-κBp50 acetylation in spinal cord tissue cultures from WT and G93A mice from 6-24 hrs and 3-6 hrs, respectively. (b) PBA increased plκB and bcl-2 proteins in a time-dependent manner. (c) PBA enhances the expression of bcl-2 mRNA in the spinal cord tissue cultures in both WT and G93A mice. (d) PBA administration in N2a cell transfected with bcl-2 reporter plasmids significantly increased bcl-2 promotor activity by 90%. (e) Curcumin, an inhibitor of NF-κB activation, significantly blocked transactivation of the bcl-2 promoter dose-dependently. *P<0.01, **P<0.0002, ***P <0.0005.

FIG. 6: κB binding site is necessary for PBA-induced bcl-2 gene transactivation. (a) WT κB bcl-2 promoter, but not site-directed κB mutant (κBm) and deletion κB mutant (ΔκB) bcl-2 promoter, is transactivated by PBA. (b) Scheme for prosurvival mechanisms of butyrates. HDAC inhibitors may act to phosphorylate lkB through the kinase pathway (30), thereby translocating NF-κBp50 to the nucleus (blue arrows). HDAC inhibitors may acetylate NF-κBp50. NF-κBp50 transactivates bcl-2 gene expression (green arrows). Upregulated bcl-2 blocks cytochrome c release (red arrow), and subsequent caspase activation and motor neuron death (black arrows). In addition, butyrates may improve histone acetylation, promoting transcription by releasing DNA template constraints (green arrows). Increase acetylation may also enhance NF-κBp50 mediated transcription. Acetylation of other transcriptioh factors and proteins may provide unknown neuroprotective effects. These transcriptional and post-translational pathways ultimately promote motor neuron survival and ameliorate the disease progression in ALS mice.

### DETAILED DESCRIPTION OF THE INVENTION

ALS remains an untreatable and uniformly lethal disease. HDAC inhibitors target multiple pathways important in ALS pathogenesis. Our data provides evidence that transcription dysfunction plays a role in the pathogenesis of ALS and suggests that therapies aimed at improving histone acetylation and transcription may provide a novel treatment strategy that translates to clinical benefits in ALS patients. The butyrates have been used in the clinic to treat patients with a number of medical conditions (References 46 and 47) and have well understood pharmacokinetics, toxicities, and side effects in man (References 46 and 47). Thus, the butyrates are compelling candidates for immediate testing in humans with ALS.

Methods

Animals: Male transgenic ALS mice of the G93A H1 strain (Jackson Laboratories, Bar Harbor, Me.) were bred with females from their background strain (B6SJLFI). Offspring were genotyped using a PCR assay on tail DNA. We have standardized criteria to ensure homogeneity of the cohorts within the testing groups. Mice were randomized from 24 litters all within 4 days from the same ‘f’ generation. Body weights were taken at 20 days and mice were equally distributed according to weight within each experimental cohort. Mice under 8 g at 20 days were excluded from the experiments. Male mice were used in the experimental paradigms, since we have observed gender differences in survival in the G93A transgenic ALS mouse model (Reference 48). These experiments were carried out in accordance with the NIH Guide for the Care and Use of Laboratory Animals, and were approved by both the Veterans Administration and Boston University Animal Care Committees.

Intraperitoneal Dosing: Based upon previous studies (Reference 29), a dose response study was performed, treating groups (n=20) of wild type and G93A mice with 200, 600, 1,200, 2,000 and 5,000 mg/kg by daily intraperitoneal injection (100 μl) of sodium butyrate (Acros Organics/Fisher Scientific, Fairlawn, N.J.) dissolved in PBS and made fresh daily. Additional groups of mice were treated in the same manner with 100, 200, 400, 600, and 800 mg/kg of phenylbutyrate (Triple Crown America, Inc. Perkasie, Pa.). Control groups of G93A mice were treated with PBS injection (vehicle). Approximately 260 mice were used for behavioral and survival analyses.

Clinical Assessment: Both motor performance and body weight were measured throughout the study. Training sessions were given on days 21 and 22 to acclimate the mice to the rotarod apparatus (Columbus Instruments, Columbus, Ohio). Motor performance (constant rotation at 16 rpm) was assessed weekly from 23-100 days of age and twice weekly from 101 days of age. Three 60 sec trials were given during each session and averaged. Body weights were recorded twice weekly at the same time of day. Gait analysis was performed measuring stride length (Reference 49).

Survival: G93A mice were assessed for morbidity and mortality twice daily, mid morning and late afternoon. Motor performance and ability to feed was closely monitored and used as the basis for determining when to euthanize the mice. The criterion for euthanization was the point in time the ALS mice were unable to right themselves after being placed on their back for 30 seconds. Some deaths occurred overnight and were recorded the following morning. Three independent observers confirmed the criterion for euthanization blinded to treatment assignment (RJF, JKK, and KS).

Neuropathological Evaluation of Sodium Butyrate and Phenylbutyrate Treatment: Beginning at 21 days, additional G93A transgenic mice and wild-type littermate control mice were treated with daily 1.2 g/kg sodium butyrate, 400 mg/kg phenylbutyrate, or PBS intraperitoneal injections. Groups of 10 animals from each treatment paradigm were deeply anesthetized and transcardially perfused with 4% buffered paraformaldehyde at 120 days of age. Forty mice were used for neuropathological analysis as previously described (Reference 29) (Supplementary Methods, below).

Stereology/Quantitation: Serial-cut lumbar spinal cord tissue-sections (n=20), beginning from L3-L5 spinal cord segments, were used for neuronal analysis. Unbiased stereological counts of ventral horn neurons were obtained from the lumbar cord in 10 mice each from NAB (1.2 g/kg), PBA (400 mg/kg), PBS-treated G93A, and wild type littermate control mice at 120 days using Neurolucida Stereo Investigator software (Microbrightfield, Colchester, Vt.) to determine the estimated number of neurons.

Acid Extraction of Histone Protein and Histone Acetylation Assay and Detection of Acetylated NF-κB p50 and Western Blotting: The detection of histone acetylation and acetylated NFκB p50 was determined as previously described (References 29 and 50) (Supplementary Methods, below).

RT/PCR Analysis: RNA extraction. After 2 weeks treatment with PBA (400 mg/kg/d) or PBS, (n=4/group) animals were sacrificed with CO2. The spinal cord was snap frozen on dry ice and total RNA extraction was immediately performed from the lumbro-sacral portion using RNeasy Lipid Tissue Mini-kit (Quiagen Inc, Valencia, Calif.). RNA was measured in a spectrophotometer at 260 nm absorbance. Fifty nanograms of RNA were used as a template for QRT-PCR amplification, using Superscript One-Step RT-PCR with platinum Taq (Invitrogen, Carlsbad, Calif.) (Supplementary Methods, below).

Promoter Activity Assay: The bcl-2 β-Gal-reporter plasmids containing the fragments from −760 to −8, −178 to −8 (κB7), −178 to −8 (κBm) that is a PCR-generated mutated κB7 site (TTTAAACACC (SEQ ID NO: 1) instead of GGGAAACACC (SEQ ID NO: 2)) and −160 to −8 were generous gift from Dr. Vincent Bours (University of Liege, Belgium) (Reference 41). The bcl-2 Luc-reporter plasmids containing the fragment from −3934 to −8 from the bcl-2 promoter was also generous gift from Dr. L. Boxer (Stanford University, California). To evaluate the ability of PBA to activate transcription of bcl-2, N2a cells were transfected by the DMRIE solution (Invitrogen, Carlsbad, Calif.) with reporter plasmids. After 24 h of incubation, cells were treated with PBA and curcumin, a know NF-κB inhibitor, harvested, and Luc or β-Gal activity in the cellular lysates were measured with a luminometer. All promoter assays were performed in duplicates. Enzyme activities were normalized to the protein concentration of the extracts.

statistics: The data are expressed as the mean±standard error of the mean. Statistical comparisons of rotarod, weight data, and histology data were compared by ANOVA or repeated measures ANOVA. Survival data was analyzed by the Kaplan-Meier survival curves. All other statistical analyses were performed using Student t-test.

TABLE 1
Table of Dose Dependent NAB- and PBA-Mediated
Survival Extension in G93A Mice

| | Dosage | Survival | Percentile | |
|---|---|---|---|---|
| Compound | (mg/kg/d) | (Days ± SD) | change | P value |
| NAB | 0 | 126.1 ± 2.7 | — | |
| | 200 | 129.2 ± 4.5 | 2.5% | P < 0.18 |
| | 600 | 141.8 ± 5.9 | 12.5% | P < 0.05 |
| | 1200 | 151.3 ± 6.8 | 20% | P < 0.01 |
| PBA | 0 | 125.7 ± 3.2 | — | |
| | 100 | 126.0 ± 4.0 | 0.2% | P < 0.27 |
| | 200 | 136.5 ± 5.5 | 8.6% | P < 0.05 |
| | 400 | 153.2 ± 6.4 | 21.9% | P < 0.001 |

Supplementary Methods

Histopathological Evaluation: Serially cut lumbar spinal cord tissue sections were stained for Nissl substance and immunostained for glial fibrilary antigen protein (GFAP), cytochrome c (Pharmingen International, San Jose Calif., dilution 1:500), activated caspase 9 and caspase 3 (Cell Signaling Tech., Inc., Beverly, Mass., dilution 1:500), acetylated histone 3 and 4 (Upstate Biotechnology, Lake Placid, N.Y., dilution, 1:1,000), plκB (dilution 1:500), NF-κB p50 (dilution 1:500), and bcl-2 (Santa Cruz Biotech, Santa Cruz, Calif., dilution 1:500), using a previously reported conjugated secondary antibody method in murine brain tissue samples. Preabsorption with excess target proteins, omission of the primary antibodies, and omission of secondary antibodies were performed to determine the amount of background generated from the detection assay.

Tissue Culture: The pharmacokinetic effect of PBA was studied by determining plκB, NF-κB, and bcl-2 expression levels using in situ tissue cultures. Freshly isolated spinal cords from wild type and G93A mice were minced and incubated with neurobasal medium for 12 hr. Tissues were subjected to 1 mM PBA for 0, 3, 6, 12, and 24 hours. After incubation, the tissues were harvested and equally divided for Western blot analysis and RT-PCR.

Acid Extraction of Histone Protein and Histone Acetylation Assay: Tissue lysate from lumbar cord was obtained by homogenizing each sample in 500 μl of PBS buffer containing 0.4M NAB, 5% Triton X-100, 100 mM PMSF, 3 mM DTT, leupeptin aprotinin, 1 mM sodium orthovanadate, 5 mM sodium fluoride, 3 mM PMSF, 3 mM DTT, 0.5 μg/ml leupeptin, and 10 μg/ml aprotinin. Two hundred μl of the lysate was washed twice with the above described 5% Triton buffer and histones were extracted by solubilization in 0.2 M HCl. The remaining 300 μl of tissue lysate was used to extract both cytoplasmic and nuclear proteins. Cytoplasmic protein pool was obtained by a centrifugation of tissue lysate at 3,000 rpm for 5 min at 4° C. Protein concentration was determined using the commercially available Coomassie Protein Assay Kit (Pierce, Rockford, Ill.). After neutralizing the pH of the acid extracted solution containing the histone pool with ammonium acetate, 15 μg of protein were separated in a 15% SDS-PAGE. Thirty micrograms of cytoplasmic and 15 μg nuclear proteins were separated in a 12% SDS-PAGE. Electrophoresed proteins were transfered to nitrocellulose membrane (Bio-Rad Laboratories, Inc., Hercules, Calif.) to proceed with Western blot analysis as follows. Briefly, membranes were blocked for non-specific antibody binding by incubation in 5% nonfat dry milk in Tris-buffered saline-Tween 20 (TBST) (50 mM TRIS HCl, pH8.0, 0.9% NaCl, 0.1% Tween 20) for 30 minutes at room temperature and incubated with primary antibodies diluted in 5% nonfat dry milk in TBST overnight at 4° C. The antibodies used were the following, acetylated histone 3 and acetylated histone 4 (Upstate Biotechnology, Lake Placid, N.Y. at 1:1000). After washing membranes in TBST, they were incubated in peroxidase conjugated IgG for 1 hour at room temperature. Membranes were washed with TBST and immunoreactive proteins were visualized by the chemiluminescence method, using the Supersignal West Pico Luminol kit (Pierce, Rockford, Ill.).

Detection of Acetylated NF-κB p50: For detection of the levels of acetylated NF-κB p50, 200 μg of nuclear extracts were diluted to a total volume of 500 microliter in lysis buffer (100 mM Tris buffer, pH7.4, containing 1% Triton X-100, 150 mM NaCl, 1 mM sodium orthovanadate, 5 mM sodium fluoride, 3 mM PMSF, 3 mM DTT, 0.5 mg/m leupeptin, and 10 mg/ml aprotinin) and incubated with 7 μl of goat anti-p50 antibody (Santa Cruz, Santa Cruz, Calif.) by shaking overnight at 4° C. Thirty-two μl of protein A-sepharose (50% v/v slurry) was added to lysates and left for 1 hr at 4° C. Beads were collected by centrifugation and washed twice with lysis buffer, once in PBS and finally resuspended with 40 μl of 2×SDS loading buffer. Samples were boiled and a 20 μl aliquot was separated in a 10% SDS-PAGE. After transferring the proteins to a nitrocellulose membrane acetylated p50 proteins were detected in Western blot analysis using an acetyl lysine-specific antibody (AbCam, Cambridge, Mass. at 1:1000).

Western Blotting: Tissue lysates and subcellular fractions were prepared using an ice-cold cell extraction buffer containing 50 mM Tris-HCl, pH 7.4, 150 mM NaCl, 2 mM EDTA, 1% Triton X-100, 1 mM phenylmethylsulfonyl fluoride (PMSF), 10 mg/ml leupeptin, 1 μM pepstatin, 1 mM N-ethylmleimide (NEM), 2 mM Na₃VO₄, 20 mM sodium pyrophosphate, and 50 mM NaF). Lysates were centrifuged at 15,000 rpm at 4° C. for 30 min. Then the protein concentration was quantified and samples were boiled for 10 min with Laemmli buffer (15 is 100 mM Tris-HCL, pH 6.8, 4% SDS, 200 mM dithiothreitol, 20% glycerol, 2% SDS, 0.2% bromophenol blue, 10 μg/ml aprotinin, 10 μg/ml leupeptin) at 100° C. In general, 30 μg of proteins were electrophoresed on 10% SDS-polyacrylamide gel and transferred to nitrocellulose membrane. Membranes were blocked in 5% skim milk in TBST (Tris, pH 7.4; 150 mM NaCl; 0.05% Tween 20) for 30 min at room temperature. Blots were probed with primary antibodies overnight at 4 ° C. The antibodies used were the following; bcl-2 (Santa Cruz Biotech, Santa Cruz, Calif.) at 1:1000, plκB (Upstate Biotechnology, Lake Placid, N.Y.) at 1:500, NF-κB p50 at 1:700, caspases 9 and caspase 3 at 1:1000, cytochrome c (BD Biosciences, San Diego, Calif.) 1:500, tubulin (Sigma, St. Louis, Mo.) at 1:3000. HRP-conjugated secondary IgG anti-Rabbit IgG and anti-mouse IgG (Bio-Rad Laboratories, Inc., Hercules, Calif.) were used at 1:5000 and 1:3000, respectively. HL-60 Cell lysates were used as positive controls (Upstate Biotechnology, Lake Placid, N.Y.).

RT/PCR Analysis: Primers were standardized in the linear range of cycles prior to onset of the plateau. The sequence of the primers is as follow:

| | |
|---|---|
| BcI2 forward: | |
| 5′-CTCGTCGCTACCGTCGTGACTTCG-3′ | (SEQ ID NO: 3) |
| BcI2 reverse: | |
| 5′-CAGATGCCGGTTCAGGTACTCAGTC-3′ | (SEQ ID NO: 4) |
| GAPDH forward: | |
| 5′-AGAGCTGAACGGGAAG-3′ | (SEQ ID NO: 5) |
| GAPDH reverse: | |
| 5′-GTTGAAGTCGCAGGAG-3′ | (SEQ ID NO: 6) |
| 18S RNA forward: | |
| 5′-CCGAGATTGAGCAATAACAGG-3′ | (SEQ ID NO: 7) |
| 18S RNA reverse: | |
| 5′-AGTTCGACCGTCTTCTCAGG-3′ | (SEQ ID NO: 8) |

The conditions of one step RT-PCR were as follow for bcl-2 primers: 30 min at 50° C., 2 min at 94° C., then 32 cycles of amplification for 15 sec at 94° C. for 15 sec, 30 sec at 68° C., 1 min at 70° C., 10 min at 72 ° C. and 4° C. GAPDH and 18 S RNA primers were done at 34 cycles and 55° C. for the annealing step. Amplified cDNA was detected by ethidium bromide staining and quantified with Chemilmager 4400 (Alpha-Innotech, San Leandro, Calif.). A bcl-2/GAPDH or bcl-2/18S ratio was calculated for each sample and an average of the four samples of each group treated with PBA or with saline solution was obtained for the comparison of the two groups. Experiments were run twice to verify the results.

N2a Cell Culture: N2a monolayer cell cultures were grown in 50% DMEM and 50% OPTI-MEM with 5% fetal bovine serum (FBS), 1% antibiotic antimycotic, and 400 microgram/ml of the neomycin analog G418. Differentiation was induced within 3 days in serum-free medium. The SOD1 mutation (G41D) was introduced into a plasmid clone of human genomic SOD1 (pHGSOD-SVneo). Stably transfected lines of N2a cells were selected with the drug G418, after transfection with SOD1 pHGSOD-SVneo plasmids. Differentiated cells were incubated without FBS for 6 days before treatments. Cells were incubated for 12 hr with 1 mM of PBA and treated with xanthine/xanthine-oxidase (X/XO) for 6hr. Cell viability was measured by MTT assay.

Immunofluorescence Staining and Microscopy: Indirect labeling methods were used to determine plκB and NF-κB p50 in N2aCells. Cells were seeded (2×10⁴ cells/ml) onto eight-well culture slides (Becton Dickinson, Bedford, Mass.) and treated with 1 mM PBA for 0.5-3 hr. The cells were washed with warm PBS and fixed at room temperature for 15 min with 4% paraformaldehyde (PFA). After washing with PBS, fixed cells were incubated with blocking solution containing 0.3% Triton X-100, 5% bovine serum albumin, and 3% goat serum for 1 hr, followed by incubation with primary antibodies overnight at 4° C. After three washes with PBS, the cells were incubated for 1 hr with FITC-conjugated (1:200 dilution) and Cy3-conjugated secondary antibody (1:200 dilution; Vector Laboratories, Burlingame, Calif.). The slides were washed three times with PBS and mounted with fluorochrome mounting solution. Images were analyzed using a fluorescence microscope. Control experiments were performed in the absence of primary antibody.

Results

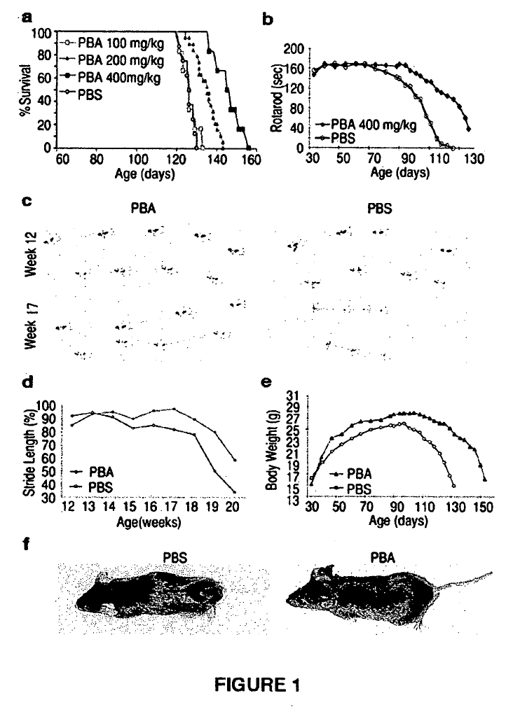

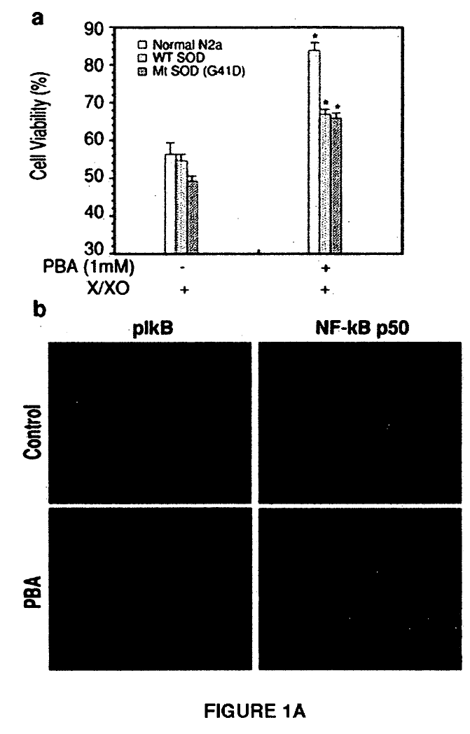

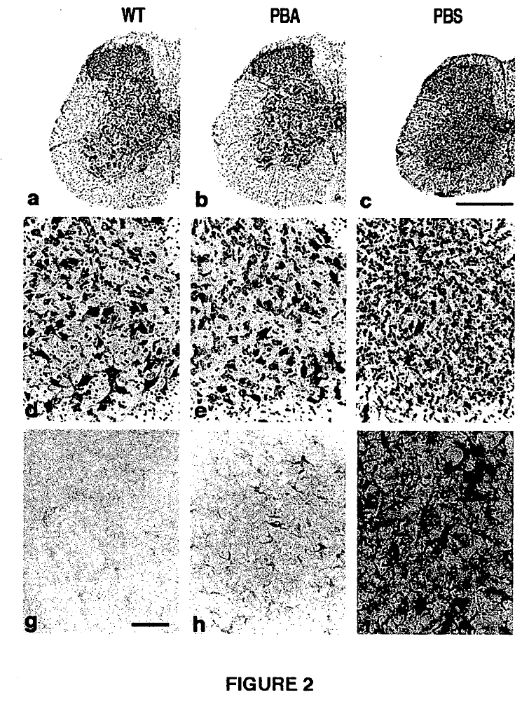

Pharmacological treatment using sodium butyrate (NAB) and phenylbutyrate (PBA) to modulate transcription significantly extended survival in a dose dependent manner, improved body weight and motor performance, and delayed the neuropathological sequelae in ALS mice (FIGS. 1-2). Intraperitoneal (i.p.) administration of NAB at 600 and 1,200, mg/kg/d significantly extended survival by 12.5% (P<0.05) and 20.0% (P<0.01), respectively, with no effects observed with i.p. NAB treatment at 200 mg/kg/d (FIG. 1a) (Table 1). At higher doses of NAB (2 g/kg/d), mice became moribund and died between 1-9 days, with sudden death occurring within 0.5-1.0 h at 4 g/kg/d. Intraperitoneal administration of PBA at 200 and 400 mg/kg/d significantly extended survival in G93A mice by 8.6% (P<0.05) and 21.9% (P<0.001), respectively (P<0.001) (FIG. 1b) (Table 1). No significant effects were observed with treatment at 100 mg/kg/d, with morbidity and mortality occurring at doses of 600 and 800 mg/kg/d. Consistent with the in vivo survival findings, pretreatment with PBA significantly protected the N2a mutant SOD (G41D) cells from death under oxidative stress conditions, using xanthine/xanthine-oxidase (Supplementary FIG. 1a). Cell viability in PBA-treated N2a/SOD1 cells was improved by 34.7% (P<0.01), in comparison to untreated cells.

Motor performance, as determined using rotarod analysis, was significantly improved after 13 wks through endstage disease (P<0.01), using the most efficacious NAB and PBA doses (FIGS. 1c and d). In addition, stride length, a selective outcome measure of motor performance and coordination, was markedly improved at 17 weeks, in comparison to untreated G93A mice (FIGS. 1f and g). Body weight loss was also significantly reduced, however, only beginning after 100 days (P<0.01), using both NAB and PBA (FIG. 1e). As previously reported in other transgenic preclinical trials using butyrates, significant differences in body weight may be an epiphenomenon of survival extension and not a primary independent event related to butyrate treatment (Reference 29).

Neuropathological analysis at 120 days of survival showed significant gross atrophy, increased astrogliosis, and neuronal loss in the lumbar spinal cord in untreated G93A mice; these findings were significantly reduced in both the NAB- and PBA-treated G93A mice (FIG. 2). Gross spinal cord atrophy in untreated G93A mice was ameliorated by both NAB (1.2 g/kg/d) and PBA (400 mg/kg/d) treatment (FIGS. 2a-d). The neuroprotective effects of NAB and PBA treatment significantly reduced the marked neuronal loss observed in untreated G93A mice (WT littermate control: 11,580.9±221.2; NAB-treated G93A mice: 9,604.4±346.2, P<0.001; PBA-treated G93A mice: 10,378.4±304.4; PBS-treated G93A mice, 4,680.6±847.4) (FIG. 2e-h). Reactive astrogliosis was a prominent finding in G93A mice spinal cord tissue sections at 120 days, and was reduced by NAB and PBA treatment in the mutant mice by comparison (FIGS. 2i-l).

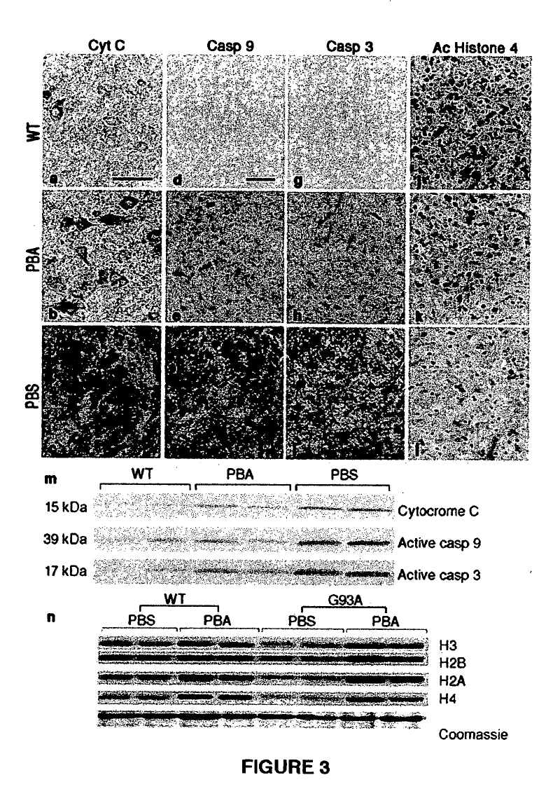

Activation of programmed cell death appears to play an important role in neuronal death in ALS. In both human and mouse ALS, a consistent event in the cascade of events leading to motor neuron is a reproducible activation of caspases. As shown in FIG. 3, butyrate ameliorated the release of cytochrome c and subsequent induction of activated caspase 9 and caspase 3 in the G93A mice (FIG. 3). Immunostaining in spinal cord tissue sections shows a dramatic reduction in cytochrome c in the cytosol of neurons in the G93A mice (FIGS. 3a-c), with reduced expression of activated caspase 9 and caspase 3 in PBA-treated G93A mice at 120 days of age (FIGS. 3d-i). These results were confirmed in Western blot analyses-(FIG. 3m).

Consistent with the hypothesis that there may be transcriptional dysregulation in ALS, we observed hypoacetylation of histones in tissue sections at 70 and 120 days of age in the G93A ALS mice, in comparison to littermate control mice. There was robust acetylated H3 and H4 immunoreactivity in spinal cord sections of wild type mice, reduced tissue H3 and H4 immunoreactivity in G93A mice, and enhanced acetylated H3 and H4 immunostaining in butyrate-treated G93A mice to near normal levels (FIGS. 3j-l, H3 data not shown). Western blot analysis confirmed the tissue section findings, showing hypoacetylation of histones in G93A mice, as compared to wild type mice, with improved H3 and H4 acetylation in butyrate-treated G93A mice (FIG. 3n).

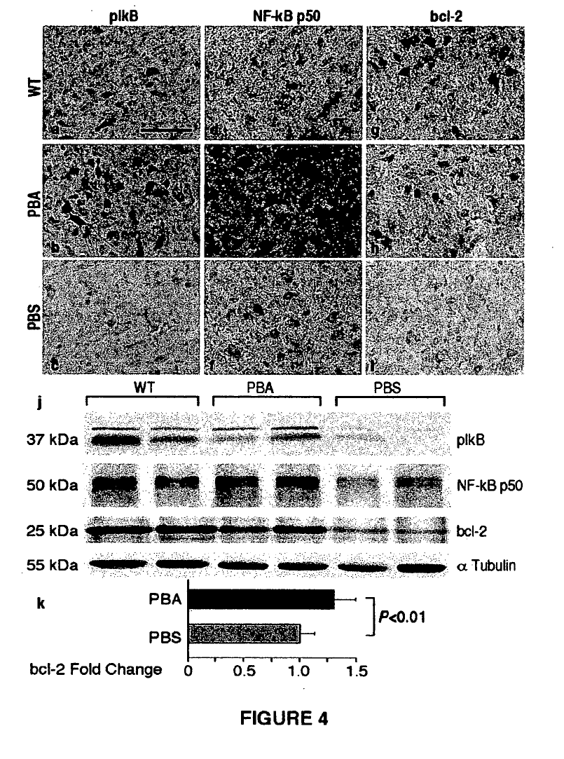

Increased histone acetylation promotes transcriptional activation and improves NF-κB/DNA binding activity. NF-κB activation through phosphorylation of lκB or directly via butyrate administration is known to regulate apoptosis (Reference 30). Here we show that immunoreactivity of both phosphorylated lκB (PlκB) and NF-κBp50 was reduced in G93A mutant mice spinal cord sections, as compared to wild type littermate control mice. In contrast, plκB and NF-κBp50 immunoreactivity was increased in the butyrate treated G93A mice (FIGS. 4a-f). These findings were present in Western blot analysis, showing significant increases in both plκB and NF-κBp50 protein expression levels in the butyrate treated G93A mice (FIG. 4j). In addition, N2a cells treated with 1 mM PBA for three hours showed increased cytosolic plκB and nuclear translocation of NF-κB p50 immunofluorescence activities (Supplementary FIG. 1b). These data provide correlate directly with the observed reduced expression of both initiation and execution elements of the apoptotic cascade and support the concept that butyrates modulate gene transcription.

NF-κBp50 can transcriptionally upregulate other anti-apoptotic genes that include bcl-2 homologs (References 18 and 22). Decreased expression of bcl-2 has been reported in ALS patients (Reference 31). In addition, overexpression of bcl-2 is neuroprotective in transgenic mutant SOD1 mice, slowing neuronal degeneration (References 17, 18 and 22). We found that bcl-2 immunoreactivity is reduced in spinal cord tissue sections from G93A mice, in comparison to littermate control mice, and that butyrate administration elevated spinal cord tissue expression of bcl-2 protein in G93A mice (FIGS. 4g-i). Western blot analysis confirmed these findings and showed a marked increase in protein levels of bcl-2 (FIG. 4j). In addition, RT-PCR showed upregulation of bcl-2 mRNA in PBA-treated G93A mice (FIG. 4k) after butyrate treatment. The bcl-2/GAPDH ratio was significantly increased in PBA-treated mutant mice by 31.4% by densitometric analysis (P<0.01).

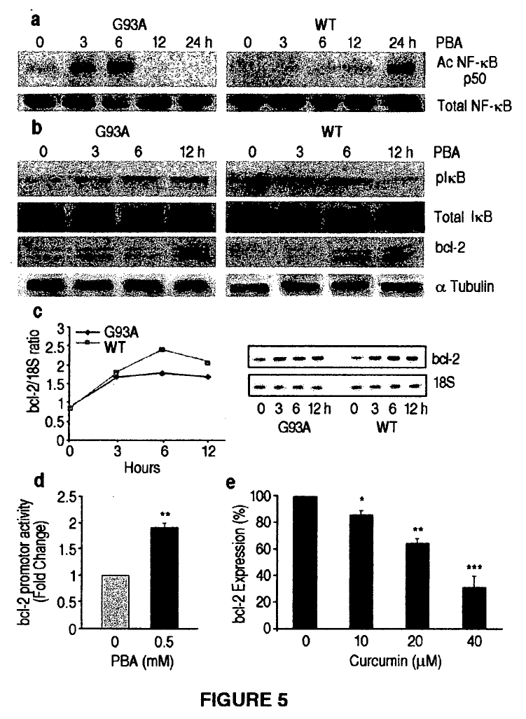

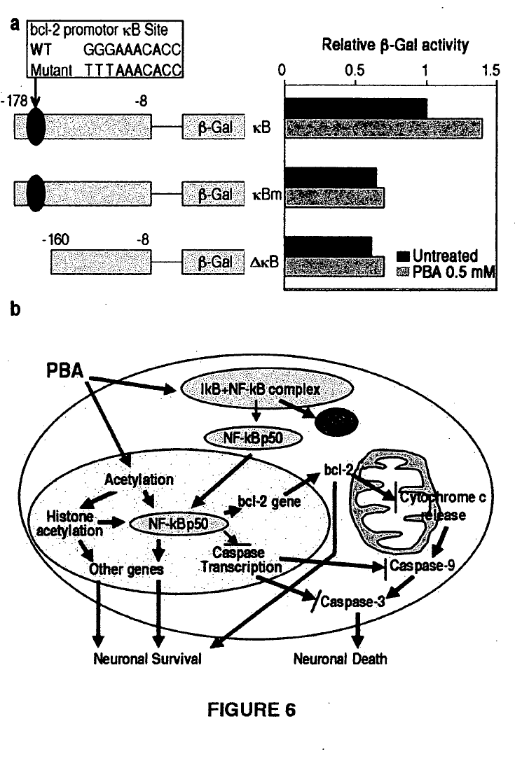

Using freshly harvested spinal cords from both wild type (WT) and G93A mice, we treated primary tissue cultures with PBA to examine the temporal expression of plκB, NF-κB p50, and bcl-2 to substantiate the in vivo data. Tissue cultures were exposed to PBA for 0, 3, 6, and 12 hours, harvested, and analyzed using Western blot and RT-PCR. Western analysis showed an increased expression of plκB, acetylated NF-κB p50, and bcl-2 in a time dependent manner (FIGS. 5a and b). The ex vivo administration of PBA resulted in mRNA upregulation of bcl-2 that was induced at 3 hours and sustained until 12 hours (FIG. 5c). In addition, N2a cells were transfected with bcl-2 reporter plasmids to evaluate whether PBA administration activated bcl-2 transcription. After 24 h of incubation, PBA administration induced bcl-2 promotor activity approximately 2 fold (FIG. 5d). Curcumin, a known inhibitor of NF-κB, dose-dependently blocked PBA-induced bcl-2 promoter activity, providing additional evidence for the induction of NF-κB as a possible mechanism of neuroprotection by butyrates (FIG. 5e). Using a transfection assay with bcl-2 promotor constructs, we show that PBA administration transactivates wild type κB site bcl-2 promotor, but not site-directed and deletion mutant kB bcl-2 promotor, suggesting that the κB site is a critical component that induces NF-κB dependant transcriptional regulation of bcl-2 gene in response to PBA (FIG. 6a).

Discussion

A definitive understanding of the molecules, events, and pathways leading to motor neuron death in ALS has not been established. A diverse variety of pathogenic events have been illuminated involving many components including: glutamatergic/excitotoxicity, formation of various aggregates of ubiquitinated proteins, perturbed mitochondrial function, impaired axonal transport, altered calcium metabolism and activation of programmed cell death cascades (Reference 14). The development of transgenic mice and rats expressing mutant forms of SOD1 has enhanced investigations of the role of these processes leading to motor neuron death. These rodent models have proven to be valuable in rational drug trials developed with candidate therapies that target the diverse postulated pathogenetic events. While the predictive value of the murine models for human ALS is not yet clear, it is noteworthy that riluzole, which extends life in humans with ALS by about 10-15%, has a roughly similar survival benefit in ALS mice. In contrast, several potential therapies that have not shown efficacy in mouse models have also been ineffective in human trials (References 32 and 33). While most preclinical mouse trials show small to moderate effects of drug compounds, arimoclomal, a heat shock protein coinducer, and the COX-2 inhibitor, celecoxib, provide robust improvement in ALS mice, extending survival by 22% and 25%, respectively (References 10 and 11). The effect of phenylbutyrate, an HDAC inhibitor, in this study is more than two-fold greater than that of riluzole in the transgenic SOD1 mice and is comparable to celecoxib in survival benefit at 22%.

HDAC inhibitors significantly improve the clinical and pathologic phenotype in models of other neurological disease (References 29 and 34). Of relevance to the present study in ALS mice, is the finding that sodium butyrate improves gene transcription and clinical symptoms in a mouse model of spinal muscular atrophy, a disorder of anterior horn cells (Reference 35). In addition, the HDAC inhibitor, Scriptaid, has been shown to reduce aggresome formation in an in vitro model expressing mutant SOD1 (Reference 36). We show, herein, that HDAC inhibitor administration, using sodium butyrate and phenylbutyrate, significantly improved the clinical and neuropathological characteristics in G93A transgenic mice, restored histone acetylation, and induced expression of plκB, NF-κBp50, and bcl-2. Ex-vivo and in-vitro experiments confirmed and expanded these findings.

NF-κB is an inducible transcription factor that plays a role in regulating inflammatory, immune, and anti-apoptotic responses in mammals, and is sequestered in the cytoplasm by its assembly with a family of inhibitory proteins, the lκBs (References 37-39). In response to a number of different stimuli, the phosphorylation of lκB results in the liberation of NF-κB, allowing the latter to translocate to the nucleus, where it becomes transcriptionally active regulating gene expression (References 37 and 40-42). HDAC inhibitors enhance NF-κB binding to DNA, augmenting gene expression (References 43 and 44). In addition, the induction of NF-κB suppresses programmed cell death by directly inhibiting caspase expression (References 37 and 40) and/or by activating bcl-2 homologs (Reference 41). Among members of the NF-κB family in mammals, the p50 subunit has been shown to be neuroprotective (Reference 39).

Increased expression of NF-κB and bcl-2 is neuroprotective in these experiments, presumably because these factors reduce levels of key components of the intrinsic apoptotic pathway (FIG. 6b). NF-κB may directly alter the transcriptional events associated with apoptosis and prevent programmed cell death (Reference 40). Alternatively, NF-κB or improved acetylation may increase transcription of bcl-2 that, in turn, blocks mitochondrial pore formation and the subsequent release of cytochrome c, resulting in a post-transcriptional event that prevents apoptosis (References 41 and 45). Acetylation may directly release constraints on the DNA template and reactivate the expression of a number of genes independently. It is unclear at this time what effect improved-acetylation may have had on other transcriptional factors and molecules in the observed neuroprotection in these studies.

While this invention has been described as having preferred sequences, ranges, steps, materials, structures, features, and/or designs, it is understood that it is capable of further modifications, uses and/or adaptations of the invention following in general the principle of the invention, and including such departures from the present disclosure as those come within the known or customary practice in the art to which the invention pertains, and as may be applied to the central features hereinbefore set forth, and fall within the scope of the invention and of the limits of the appended claims.

### REFERENCES

The following references, and those cited in the disclosure herein, are hereby incorporated herein in their entirety by reference.
1. Rowland, L. & Shneider, N. Amyotrophic lateral sclerosis. N. Engl. J. Med. 344,1688-1700 (2001).
2. Bensimon G, Lacomblez L, Meininger V & the ALS/Riluzole Study Group. A controlled trial of Riluzole in amyotrophic lateral sclerosis. N. Eng. J. Med. 330, 585-591(1994).
3. Miller, R. G, Mitchell, J. D., Lyon, M. & Moore, D. H. Riluzole for amyotrophic lateral sclerosis (ALS)/motor neuron disease (MND). Amyotroph. Lateral Scler. Other Motor Neuron Disord. 4, 191-206 (2003).
4. Rosen, D. R. et al. Mutations in Cu/Zn superoxide dismutase are associated with familial amyotrophic lateral sclerosis. Nature 362, 59-62 (1993).
5. Brown, R. H. Jr, & Robberecht, W. Amyotrophic lateral sclerosis: pathogenesis. Semin. Neurol. 21,131-139 (2001).
6. Andersen, P. M. et al. Sixteen novel mutations in the Cu/Zn superoxide dismutase gene in amyotrophic lateral sclerosis: a decade of discoveries, defects and disputes. Amyotroph. Lateral Scler. Other Motor Neuron Disord. 4, 62-73 (2003).
7. Gurney M. F. et al. Motor neuron degeneration in mice that express a human Cu,Zn superoxide dismutase mutation. Science 264, 1772-1775 (1994).
8. Ripps, M. E., Huntley, G. W., Hof, P. R., Morrison, J. H. & Gordon, J. W. Transgenic mice expressing an altered murine superoxide dismutase gene provide an animal model of amyotrophic lateral sclerosis. Proc. Natl. Acad. Sci. USA 92, 689-693 (1995).
9. Bruijn, L. et al. ALS-linked SOD1 mutant G85R mediates damage to astrocytes and promotes rapidly progressive disease with SOD1-containing inclusions. Neuron 18, 327-338 (1997).
10. Drachman, D. B. et al. Cyclooxygenase 2 inhibition protects motor neurons and prolongs survival in a transgenic mouse model of ALS. Ann. Neurol. 52, 771-778 (200).
11. Kieran, D. et al., Treatment with arimoclomol, a coinducer of heat shock proteins, delays disease progression in ALS mice. Nat. Med. 10, 402-405 (2004).
12. Klivenyi, P. et al. Neuroprotective effects of creatine in a transgenic animal model of amyotrophic lateral sclerosis. Nat. Med. 5, 347-350 (1999).
13. Zhu S. et al. Minocycline inhibits cytochrome c release and delays progression of amyotrophic lateral sclerosis in mice. Nature 417, 74-78 (2002).
14. Cleveland, D. W. & Rothstein, J. D. From Charcot to Lou Gehrig: Deciphering selective motor neuron death in ALS. Nat. Neurosci. Rev. 2, 806-819 (2001).
15. Pasinelli, P., Houseweart, M., Brown, R. & Cleveland, D. Caspase-1 and -3 are sequentially activated in motor neuron death in Cu/Zn superoxide dismutase mediated familial amyotrophic lateral sclerosis. Proc. Natl. Acad. Sci. USA 97, 13901-13906 (2000).
16. Friedlander, R. M. Apoptosis and caspases in neurodegenerative diseases. N. Engl. J. Med. 348,1365-1375 (2003).
17. Kostic, V., Jackson-Lewis, V., de Bilbao, F., Dubois-Dauphin, M. & Przedborski, S. Bcl-2: Prolonging Life in a Transgenic Mouse Model of Familial Amyotrophic Lateral Sclerosis. Science 277, 559-562 (1997).
18. Vukosavic, S. et al. Delaying caspase activation by Bcl-2: A clue to disease retardation in a transgenic mouse model of amyotrophic lateral sclerosis. J. Neurosci. 20, 9119-9125 (2003).
19. Wakabayashi , K. et al. Bcl-2 related proteins are dramatically induced at the early stage of differentiation in human liver cancer cells by a histone deacetylase inhibitor projecting an anti-apoptotic role during this period. Oncol. Rep. 7, 285-288 (2000).
20. Malaspina, A., Kaushik, N. & de Belleroche, J. Differential expression of 14 genes in amyotrophic lateral sclerosis spinal cord detected using gridded cDNA arrays. J. Neurochem. 77, 132-145 (2001).
21. Ishigakia, S. et al. Differentially expressed genes in sporadic amyotrophic lateral sclerosis spinal cords screening by molecular indexing and subsequent CDNA microarray analysis. FEBS Lett. 531, 354-358 (2002).
22. Gonzalez de Aguilar, J. G. et al. Alteration of the Bcl-x/Bax ratio in a transgenic mouse model of amyotrophic lateral sclerosis: evidence for the implication of the p53 signaling pathway. Neurobiol. Disease 7, 406-415 (2000).
23. Yoshihara, T. et al. Differential expression of inflammation-and apoptosis-related genes in spinal cords of a mutant SOD1 transgenic mouse model of familial amyotrophic lateral sclerosis. J. Neurochem. 80, 158-167 (2002).
24. Olsen, M. K. et al. Disease mechanisms revealed by transcription profiling in SOD1-G93A transgenic mouse spinal cord. Ann. Neurol. 50, 730-740 (2001).
25. Dangond, F. et al. The molecular signature of late-stage human ALS revealed by expression profiling of post-mortem spinal cord gray matter. Physiol. Genomics 16, 229-239 (2004).
26. Van Lint, C., Emiliani, S. & Verdin, E. The expression of a small fraction of cellular genes is changed in response to histone hyperacetylation. Gene Expr. 5, 245-253 (1996).
27. Vigushin, D. M., Coombes, R. C. Histone deacetylase inhibitors in cancer treatment. Anticancer Drugs 13, 1-13 (2002).
28. Cremer J E, Lai J C, Sarna G S. Rapid blood-brain transport and metabolism of butyrate and pyruvate in the rat after portocaval anastomosis. J. Physiol. 266, 70P-71P (1977).
29. Ferrante, R. J. et al. Histone deacetylase inhibition by sodium butyrate chemotherapy ameliorates the neurodegenerative phenotype in Huntington's disease mice. J. Neurosci. 23, 9418-9427 (2003).
30. Mayo, M. W. et al. Ineffectiveness of histone deacetylase inhibitors to induce apoptosis involves the transcriptionaI activation of NF-□B through the Akt pathway. J. Biol. Chem. 278, 18980-18989 (2003).
31. Mu, X., He, J., Anderson, D. W., Trojanowski, J. Q., & Springer, J. E. Altered expression of bcl-2 and bax mRNA in amyotrophic lateral sclerosis spinal cord motor neurons. Ann. Neurol. 40, 379-386 (1996).
32. ALS CNTF Study Group. A double-blind placebo-controlled clinical trial of subcutaneous recombinant human ciliary neurotrophic factor (rhCNTF) in amyotrophic lateral sclerosis. Neurol. 46,1244-1249 (1996).
33. Cudkowicz, M. et al. A randomized, placebo-controlled trial of topiramate in amyotrophic lateral sclerosis. Neurology 61, 456464 (2003).
34. Steffan, J. S. et al. Histone deacetylase inhibitors arrest polyglutamine-dependent neurodegeneration in Drosophila. Nature 413, 739-743 (2001).
35. Chang, J-G. et al. Treatment of spinal muscular atrophy by sodium butyrate. Proc. Natl. Acad. Sci. USA 98, 9808-9813 (2001).
36. Corcoran, L. J., Mitchison, T. J. & Liu, Q. A novel action of histone deacetylase inhibitors in a protein aggresome disease model. Curr. Biol. 14, 488-492 (2004).
37. Wang, C-Y., Mayo, M. W. & Baldwin, A. S. Jr. TNF- and cancer therapy-induced apoptosis: Potentiation by inhibition of NF-kappaB. Science 274, 784-787 (1996).
38. Kassed, C. A., Willing, A. E., Garbuzova-Davis, S., Sanberg, P. R. & Pennypacker, K. R. Lack of NF-kappaB p50 exacerbates degeneration of hippocampal neurons after chemical exposure and impairs learning. Exp. Neurol. 176, 277-288 (2002).
39. Pennypacker, K. R., Kassed, C. A., Eidizadeh, S., Saporta, S. & Sanberg, P. R. NF-□B p50 is increased in neurons surviving hippocampal injury. Exp. Neurol. 172, 307-319 (2001).
40. Beg, A. A. & Baltimore, D. An essential role for NF-kappaB in preventing TNF-alpha-induced cell death. Science 274, 782-784 (1996).
41. Viatour, P. et al. NF-kappa B2/p100 induces Bcl-2 expression. Leukemia 17, 1349-1356 (2003).
42. Bowie, A. & O'Neil, L. A. J. Oxidative stress and nuclear factor-□B activation. A reassessment of the evidence in the light of recent. Biochem. Pharmacol. 59, 13-23 (2000).
43. Chen, L., Fischle, W, Warner, V. E. & Greene, C. Duration of nuclear NF-kB action regulated by reversible acetylation. Science 293, 1653-1657 (2001).
44. Glaser, K. B. et al. Gene expression profiling of multiple histone deacetylase (HDAC) inhibitors: defining a common gene set produced by HDAC inhibition in T24 and MDA carcinoma cell lines. Mol. Cancer Ther. 2, 151-163 (2003).
45. Heckman, C. A., Mehew, J. W. & Boxer, L. M. NF-kappaB activates Bcl-2 expression in t(14;18) lymphoma cells. Oncogene 21, 3898-3908 (2001).
46. Gilbert, J. et al. A Phase I dose escalation and bioavailability study of oral sodium phenylbutyrate in patients with refractory solid tumor malignancies. Clin. Cancer Res. 7, 2292-2300 (2001).
47. Warrell, R. P. et al. Therapeutic targeting of transcription in acute promyelocytic leukemia by use of an inhibitor of histone deacetylase. J. Natl. Cancer Inst. 90, 1621-1625 (1998).
48. Ferrante, R. J., Klein, A. M., Dedeoglu, A. & Beal, M. F. Therapeutic efficacy of Ebb761 (Gingko Biloba Extract) in a transgenic mouse model of amyotrophic lateral sclerosis. J. Mol. Neurosci. 17, 89-96 (2001).
49. Barneoud, P. & Curet, O. Beneficial effects of lysine acetylsalicylate, a soluble salt of aspirin, on motor performance in a transgenic model of amyotrophic lateral sclerosis. Exp. Neurol. 155, 243-251 (1999).
50. Ryu, H. et al. Histone deacetylase inhibitors prevent oxidative neuronal death independent of expanded polyglutamine repeats via an Sp1-dependent pathway. Proc. Natl. Acad. Sci. USA 100, 4281-4286 (2003).

What is claimed is:

1. A method of ameliorating or abrogating the effects of a neurodegenerative disorder in a subject, comprising:
administering a histone deacetylase (HDAC) inhibiting agent in a subject in need thereof;
wherein the HDAC inhibiting agent comprises a butyrate.

2. The method of claim 1, wherein:
the butyrate is selected from the group consisting of sodium butyrate, and sodium phenylbutyrate.

3. The method of claim 2, wherein:
the neurodegenerative disorder comprises amyotrophic lateral sclerosis (ALS).

4. A method of preventing neuronal death in a subject having amyotrophic lateral sclerosis, comprising:
administering a histone deacetylase (HDAC) inhibiting agent in a subject in need thereof;
wherein the HDAC inhibiting agent comprises a butyrate.

5. The method of claim 4, wherein:
the butyrate is selected from the group consisting of sodium butyrate, and sodium phenylbutyrate.

6. The method of claim 4, wherein:
the HDAC inhibiting agent is administered intraperitoneally.

7. A method of treating a subject having amyotrophic lateral sclerosis, comprising:
administering a histone deacetylase (HDAC) inhibiting agent in a subject in need thereof;
wherein the HDAC inhibiting agent comprises a butyrate.

8. The method of claim 7, wherein:
the butyrate is selected from the group consisting of sodium butyrate, and sodium phenylbutyrate.

9. The method of claim 7, wherein:
the HDAC inhibiting agent is administered intraperitoneally.

10. A method of protecting neural cells in a subject having a neurological disorder, comprising:
increasing histone acetylation by administering a butyrate in a subject in need thereof.

11. The method of claim 10, wherein:
the butyrate is selected from the group consisting of sodium butyrate, and sodium phenylbutyrate.

12. The method of claim 10, wherein:
the neurological disorder comprises amyotrphic lateral sclerosis.

13. A method of inducing expression of plκB in a subject, comprising:
administering a histone deacetylase (HDHC) inhibiting agent in a subject in need thereof; and
wherein the HDAC inhibiting agent comprises a butyrate.

14. The method of claim 13, wherein:
the butyrate is selected from the group consisting of sodium butyrate, and sodium phenylbutyrate.

15. A method of inducing expression of NF-κB p50 in a subject, comprising:
a) administering a histone deacetylase (HDHC) inhibiting agent in a subject in need thereof; and
b) wherein the HDAC inhibiting agent comprises a butyrate.

16. The method of claim 15, wherein:
the butyrate is selected from the group consisting of sodium butyrate, and sodium phenylbutyrate.

17. A method of inducing expression of bcl-2 in a subject, comprising:
administering a histone deacetylase (HDHC) inhibiting agent in a subject in need thereof; and
wherein the HDAC inhibiting agent comprises a butyrate.

18. The method of claim 17, wherein:
the butyrate is selected from the group consisting of sodium butyrate, and sodium phenylbutyrate.

19. The method of claim 1, wherein:
the subject comprises a mammal.

20. The method of claim 3, wherein:
the subject comprises a transgenic mouse model of ALS.
